(19) United States
(12) Patent Application Publication
(10) Pub. No.: US 2007/0066987 A1
(43) Pub. Date: Mar. 22, 2007

(54) Bone navigation probes

(76) Inventors: Donald L. Scanlan JR., Huntingdon Valley, PA (US); Dean Allen Entrekin, Downingtown, PA (US); Michael W. Paris, Lansdale, PA (US); Charanpreet S. Bagga, Phoenixville, PA (US); Matthew Thomas Hollenbeck, Conshohocken, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/223,085

(22) Filed: Sep. 9, 2005

(51) Int. Cl. A61B 17/32; A61B 17/34; A61B 10/00

(52) U.S. Cl. 606/184; 600/564; 600/567; 606/185

(57) ABSTRACT

Novel devices and systems are provided to provide safe navigation through cancellous bone marrow spaces attendant to the harvesting of bone marrow. In accordance with a featured embodiment, these comprise a rigid, self re-directing stylet having a proximal, mating end for cooperating with a cannula handle together with a distal end having a convex geometry, such as a dome.

### FIELD OF THE INVENTION

The present invention provides self re-directing stylets or, more generally, probes for navigating within cancellous bone enclosed by relatively dense cortical bone. Also provided are methods for using the stylets in helping to retrieve bone marrow aspirate. Methods for using the stylets in procedures involving delivery of material are also provided.

### BACKGROUND OF THE INVENTION

Bone marrow aspiration is a common surgical procedure that involves harvesting of bone marrow from bony structures such as the iliac crest and vertebral body. As will be appreciated, relatively soft, relatively porous, cancellous bone generally resides within relatively hard and less porous cortical bone in areas such as the iliac crest. Access to the cancellous bone permits the harvesting of appreciable amounts of marrow, which is useful in a number of important procedures and in research. Typically, for these procedures, bone marrow aspiration needles are inserted by either tapping or manually advancing into place through the cortical bone and into the cancellous bone area. These needles often include a modular outer cannula and sharp tip stylet assembly, or a one-piece cannulated needle with a sharpened distal tip that is positioned through the cortical wall into the middle cancellous region of the bone, where the marrow is to be harvested. In the case of the assembly, the stylet is removed from the cannula to draw the marrow outside of the body through the cannula, usually into a syringe. In the case of the one piece needle, side ports or fenestrations may be present on the needle to allow for aspiration through the cannula. During the harvesting procedure, the needle is maneuvered within the cancellous bone space to access the marrow.

There are numerous devices in the art that allow for the aspiration of bone marrow from bone. As a class, they suffer from a significant shortcoming in that the sharpened component is capable not only of penetrating a first cortical bone area and into the cancellous bone mass, but also of passing through the cancellous bone and through a further cortical bone wall and out of the bone entirely. The resulting injury can be severe and is clearly to be avoided.

None of the prior devices for marrow harvest, however, have a safety feature for reducing the risk of or preventing damage to tissue external to bone from incorrect or inappropriate employment of the devices. There is a need to provide a device for bone marrow aspiration, which can be used to probe or navigate through cancellous bone safely with little or no risk of unintended puncture to the cortical bone. Further, there is a need in the art to provide a system for bone marrow aspiration that includes a bone navigating probe having improved safety features.

### SUMMARY OF THE INVENTION

The present invention provides a solid, effectively rigid, self re-directing probe or stylet for navigating cancellous bone. The probe is used as part of an assembly, which is able to pierce cortical bone, such as of the iliac crest, through employment of a sharp stylet. An effectively rigid navigating stylet comprising a proximate mating end and a distal end having a convex geometry is then employed to explore the cancellous bone space within the surrounding cortical bone envelope. The invention thus provides a bone navigation system comprising a cannula, a sharp tip stylet, and a rigid navigating stylet. The cannula has a proximate end and a distal end. The proximate end of the cannula comprises a handle assembly having finger recesses, securing mechanism, such as a Luer to dock a syringe, and preferably a locking arrangement such as a locking recess or notch for receiving a locking tab on a mating stylet. The distal end has fenestrations. The sharp tip stylet has a hub assembly preferably comprising a locking tab and a hammering surface that mates with a handle in an overall assembly. The rigid navigating stylet for navigating trabecular bone may feature a hub assembly, such as one comprising a locking tab on the proximate end of the stylet and a convex geometry, such as a dome, on the distal end.

The cannula and stylets and, indeed, the entire assembly may be formed from any material consistent with the intended functions of the respective parts. Stainless steel is conventional and convenient for medical devices and is one preferred material. A number of plastics, polymers, metals and rubbers which are relatively rigid and can be sterilized may also be employed, especially for the portions of the device which are not intended to enter the bone spaces. The sharp portions of the device, especially the distal portions of the cannula and sharp tip stylet are preferably metal, especially stainless steel. Certain plastics and ceramics can be made to hold a sharp edge and could be employed, however, for these aspects of the invention. In one embodiment, the cannula bears graduated markings over at least a portion of its length to help gauge depth or location. Other portions may also carry geometric landmarks or markings to facilitate navigation in the bone. The landmarks may be at least partially radiopaque so that they may be monitored via fluoroscopy. The several handle and hub assemblies may comprise plastic, metal or other convenient materials.

Also provided are methods of accessing a trabecular site using the rigid navigating stylet. The method comprises piercing cortical bone with the sharp tip stylet preferably having a hub assembly comprising a locking tab and a striking surface that mates with a handle assembly of the cannula, also referred to as a docking cannula. Once the outer cortical bone is pierced, the sharp tip stylet is separated and removed from the cannula. The navigating stylet is then inserted into the lumen of the cannula for navigating the assembly in the cancellous bone. The navigating stylet preferably has a hub assembly comprising a locking tab on the proximate end and a convex geometry, such as a dome, hemi-sphere or spherical portion, on the distal end. The methods disclosed herein may also be used to access the iliac crest, pedicle or other such suitable bone during aspiration of bone marrow. It will be appreciated that further embodiments of the present invention may be developed from this disclosure.

### BRIEF DESCRIPTION OF THE DRAWINGS

FIG. 1 is an illustration of the three components of an exemplary bone marrow aspiration assembly: a hollow cannula (1a), a partial side view of the sharp tip stylet (1b), and a front view of the navigating stylet (1c). Note that 140 is the same hub design—size and shape, for both the sharp tip stylet and the navigating stylet.

FIG. 2 is an illustration of a navigating stylet and cannula assembly.

FIG. 3 is an enlarged cross-sectional view of an assembled handle for the assembly showing the mechanically interlocked tab of the stylet hub and the clearance recess space created between the cannula Luer and stylet hub.

FIG. 4 is an enlarged view of the distal end of the navigating stylet shown inserted into the cannula with fenestrations apparent. The convex tip geometry is depicted. Alternative geometries are shown in FIGS. 4a, 4b and 4c.

FIG. 5 shows an embodiment of the system of the present invention inserted into an iliac crest.

FIGS. 5a and 5b show a sharp tip stylet puncturing the cortical bone and a navigating stylet of this invention which has re-directed off the cortical wall back into cancellous space.

FIG. 6 depicts use of the navigation stylet and other aspects of this invention for securing access to vertebral spaces with improved safety.

### DESCRIPTION OF CERTAIN PREFERRED EMBODIMENTS

The present invention relates to apparatuses for safely probing cancellous bone while avoiding the accidental penetration of surrounding cortical bone. A preferred embodiment of the invention includes a solid, effectively rigid, self re-directing or navigating stylet for probing intraosseous spaces. As used herein, the term intraosseous refers to the bony area between two cortical walls. This navigating stylet comprises a proximate mating end for mating with a cannula, and a distal end in the shape of a convex geometry, such as a dome, hemi-sphere or partial spherical portion. An embodiment of the invention may also be described as an effectively rigid, self re-directing stylet for navigating trabecular bone. After the cortical bone of the iliac crest, pedicle or other such bone is pierced using a cannula- sharp tip stylet assembly, the stylet comprising a proximate mating end and a “blunt” end, one which is convex by reference to the plane normal to the axis of the stylet is inserted and maneuvered via the cannula. In one embodiment, the end is in the shape of a dome. The convex end at least partially enables the stylet to be self-redirecting. As such, this end is a safety feature.

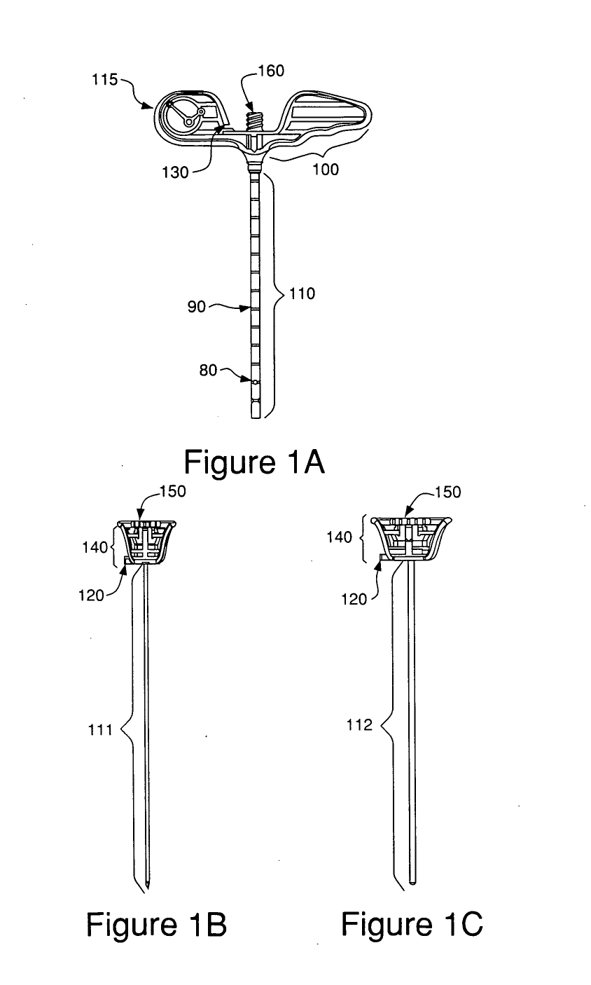

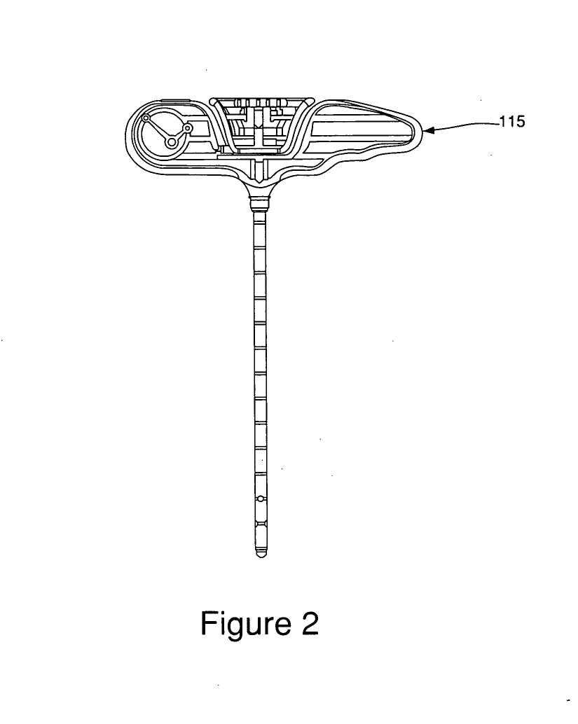

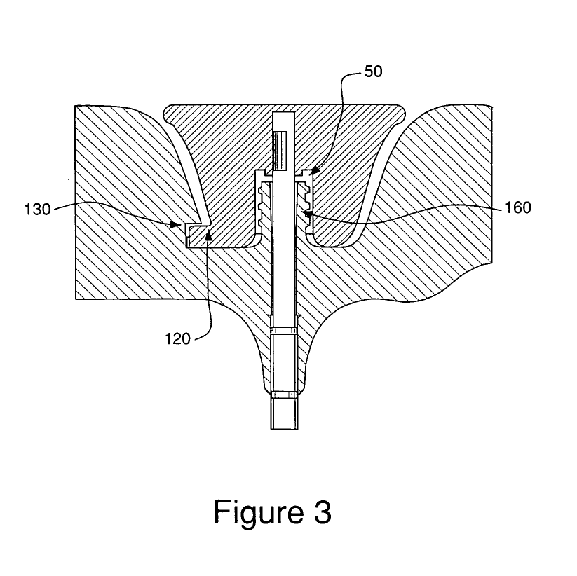

An exemplary, novel device for the aspiration of bone marrow is disclosed in FIGS. 1 through 6. A three part assembly can be seen in FIGS. 1A, 1B, and 1C. This embodiment comprises a hollow cannula 110, a sharp tip stylet 111 and a navigating stylet 112. The system allows a user to gain access to, and safely navigate within, a cancellous bone space during a bone marrow aspiration procedure.

While selection of dimensions is within the purview of persons skilled in the art, the length of the sharp tip stylet 111 and navigating 112 stylet is generally between about 12 cm and about 18 cm; the diameter can range between about 2 mm and about 12 mm. The length of cannula 110 is generally between about 10 cm and about 15 cm. It should be noted that the respective lengths of the stylets should always be longer than the length of the cannula for best function. The inner diameter of the cannula is slightly larger than the outer diameter of the stylets, allowing the components to operate as a system during the advancement of the assembly into bone in a generally co-axial fashion. The distal end of the cannula 110 preferably has a conical grind 61 that gradually tapers from the outer diameter of the cannula to its inner diameter, which is slightly larger than the outer diameter of the stylet 111 and 112. The materials, structure, design and other aspects of the cannula—and, indeed, the remaining portions of the system provided hereby, will be guided by the functions to be performed thereby. Thus, in the case of the cannula, a metal suitable for surgery, especially stainless steel, is preferably employed. The dimensions of the cannula-sharp tip stylet assembly are selected to permit transmission of appropriate force for penetrating the cortical bone.

The present invention also provides bone marrow aspiration assemblies. These devices allow the user to safely navigate within the cancellous bone regions in order to safely obtain bone marrow aspirate. A cannula interfaces, preferably co-axially, with both a sharp tip stylet and a navigating stylet. A cannula handle, especially one which is ergonomically designed for user comfort and careful control of the stylets is also preferably provided as part of the system. The cannula handle may also be seen to allow for stability and ease of use when applying force to the cannula-sharp tip stylet assembly. It also preferably is adapted, such as via a preferred, threaded Luer connection with an inlet, to provide a docking station for the stylets to be inserted and withdrawn from the cannula and for a surgical syringe to withdraw bone marrow. The stylets preferably have a proximal hub composed of a rigid medical grade plastic. The flat surface of the stylet hub provides a stable platform if hammering is necessary to perforate the cortical bone with the sharp tip stylet. The cannula Luer, shown infra as 160 is preferably protected by a recess 50 that is within the hub assembly 140.

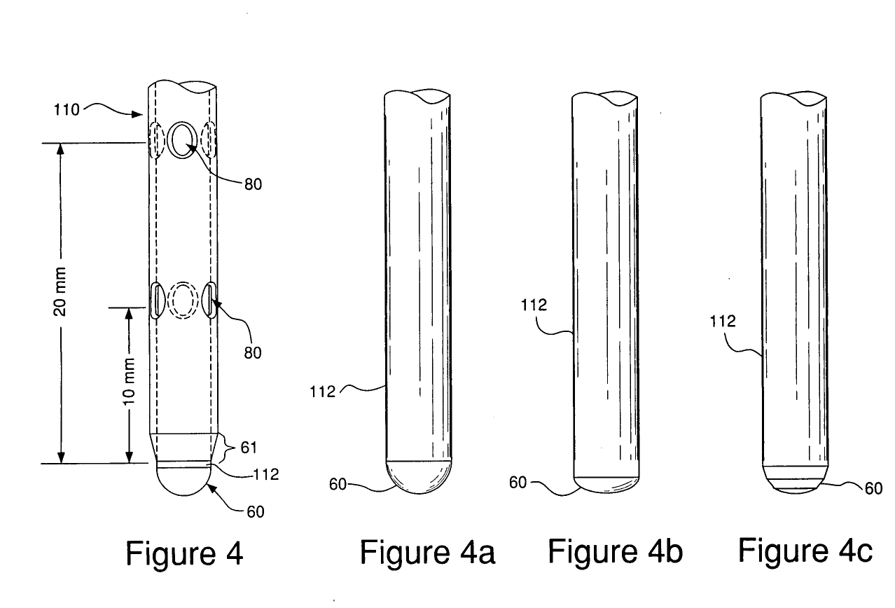

The exemplary cannula handle assembly 115 preferably has finger recesses 100 which provide comfort and handling stability for the user. The placement of the finger recesses 100 allows the user to grip the handle 115 in a variety of configurations. The cannula 110 can be gripped between the index and middle, or middle and ring fingers. The overall size of the handle 115 is designed to fit in the palm of the hand and rounded edges are provided to confer a comfortable grip. In the cannula 110 medical grade stainless steel is insert-molded into the cannula handle assembly 115, which is composed of a rigid medical grade plastic. Laser etched graduations 90 spaced, e.g. 1 cm apart allow for depth measurement within the cancellous bone spaces. The stylets are secured in the assembly by a notch 130 in the cannula handle 115. A standardized Luer 160 is inset within the cannula handle to allow attachment of a surgical syringe for bone marrow aspiration and also to serve as an inlet port for the insertion of the stylets into the lumen of the cannula 110. Fenestrations 80—holes—at the distal end of the cannula 110 allow bone marrow to be aspirated from different directions and depths, even when the cannula is in a stationary position. This feature also allows a relatively large volume of bone marrow to be harvested at each location compared to non-fenestrated designs. There are preferably at least three fenestrations 80 located e.g. within 20 mm from the distal end of the cannula 110 and radially spaced 120° apart on the same cross-sectional plane as shown in FIG. 4. In this embodiment, a second set of three fenestrations 80 are located 10 mm from the distal end of the cannula 110 on the same cross-sectional plane and are conveniently, radially offset by 90° from the fenestrations 80 located e.g. 20 mm from the distal end of the cannula 110. It should also be noted that the shape of the fenestrations can be of a variety shapes including circles or ovoids, but are preferably oval shape for allowing maximum bone marrow to be aspirated while still maintaining the mechanical integrity of the cannula.

The stylets 111, 112 may comprise exemplary medical grade stainless steel insert-molded into a rigid plastic hub 140. The stylet hubs 140 may be color coded to let the user know which stylet is inserted in the cannula 110. The sharp tip stylet hub 140 may be black or red, indicating danger or caution. The blunt tip navigating stylet hub 140 is preferably green to indicate that the safe “navigation” tip is in place. It should be understood that the sharp tip stylet may be a diamond tip or trocar stylet, or may be beveled. The sharp tip stylet could also be like a cork screw or may even have threads or cutting flutes like a drill bit. When assembled with the cannula handle 115, the hubs 140 provide a stable hammering platform 150 for perforating cortical bone with the trocar stylet 111. The hub 150 is designed to fit securely into the cannula handle 115 through a locking mechanism. When a stylet of the present invention is fully inserted into the lumen of the cannula 110, the tab 120 of the stylet hub 150 locks into a notch 130 in the cannula handle 115 through a twisting motion. This locking mechanism prevents separation of the stylet from the cannula 110 and forms a hammering surface 150. The ergonomic design of this handle assembly 115 provides stability and ease of use when exerting force on the needle 111 to perforate the cortical bone.

The recess 50 in the stylet hub 140, provides a protective housing for the cannula Luer 160. This housing prevents damage to the Luer threads if hammering is necessary to perforate the cortical bone with the trocar stylet 111. Once the trocar stylet 111 has been used to pierce the cortical wall, it is easily removed by rotating the stylet hub 140 to disengage the tab 120 from the recess 130. The trocar stylet 111 can then be withdrawn from the cannula 110 and exchanged with the navigating stylet 112, which is inserted and locked into place using the tab 120 by rotating the stylet hub 140.

The navigating stylet 112 allows the user to safely navigate within the cortical bone boundaries in order to correctly position the cannula 110 within the cancellous bone marrow space. A convex end 60, one which is preferably rounded, chamfered or otherwise blunted is provided on the navigating stylet so that the tip will deflect off of dense, cortical bone walls which surround the softer cancellous bone regions, thus re-directing the cannula assembly within the cancellous bone marrow space. This minimizes the risk for accidental perforation of the cortical bone boundaries, which could result in damage to nerves, vessels, or soft tissue structures.

FIG. 4a depicts the distal tip 60 of a navigating stylet 112 of the invention apart from the cannula. The tip may be seen to be blunt and convex with respect to the plane normal to the axis of the stylet. In FIG. 4a, the convexity is essentially that of a portion of a sphere. It will be understood, however, that other convexities may also provide sufficient bluntness and, especially, roundness as to give rise to the benefits conferred by this invention. Thus, FIG. 4b shows a shape which is an ovoid solid rather than a portion of a sphere. Multiple bevels or chamfers shown in FIG. 4c may also be employed to achieve the convexity contemplated by the present invention and all persons of skill in the art will appreciate the generality of the term convex in this context.

Once the desired depth or location is reached, the navigating stylet 112 can be removed, e.g. by rotating the stylet hub 140 to disengage the tab 120, and withdrawing the stylet 112 out of the cannula 110. To aspirate bone marrow, a surgical syringe (not shown) may conveniently be attached to the cannula Luer 160 and suction applied by withdrawing the syringe plunger. Prior to repositioning or removing the cannula 110, the navigating stylet 112 is preferably reinserted and locked into place via the locking mechanism of the hub 140. If it is necessary to harvest from an additional site, the trocar stylet 111 may be re-inserted to perforate the cortical bone and gain entry into the cancellous bone space at another location.

While one preferred embodiment of the device has been disclosed, the invention is not limited to these specifications. For example, while the preferred length of the stylet is between 12 and 18 centimeters, and the preferred length of the cannula is between 10 and 15 centimeters, any functional length up to and including 25 centimeters may be necessary. Further, while the diameter of the stylet has been disclosed as being between 2 and 12 millimeters, the utility of a smaller or larger device can be useful. In addition, the cannula and stylets have been disclosed as being composed of stainless steel, however these components may be made of any medical grade material that has the strength and rigidity to perforate through cortical bone and navigate within the marrow space. Further, while the cannula handle and stylet hubs have been disclosed as being constructed out of plastic, other rigid medical grade materials could be used for the same purpose. While one locking mechanism for securing the stylet into the cannula handle has been disclosed, any method of mechanically interlocking the stylet to the cannula that allows easy insertion and removal of the stylet is acceptable. The cannula handle has been disclosed as having a Luer as the inlet port to allow for the attachment of a surgical syringe, however other coupling devices such as press fit ports could be used.

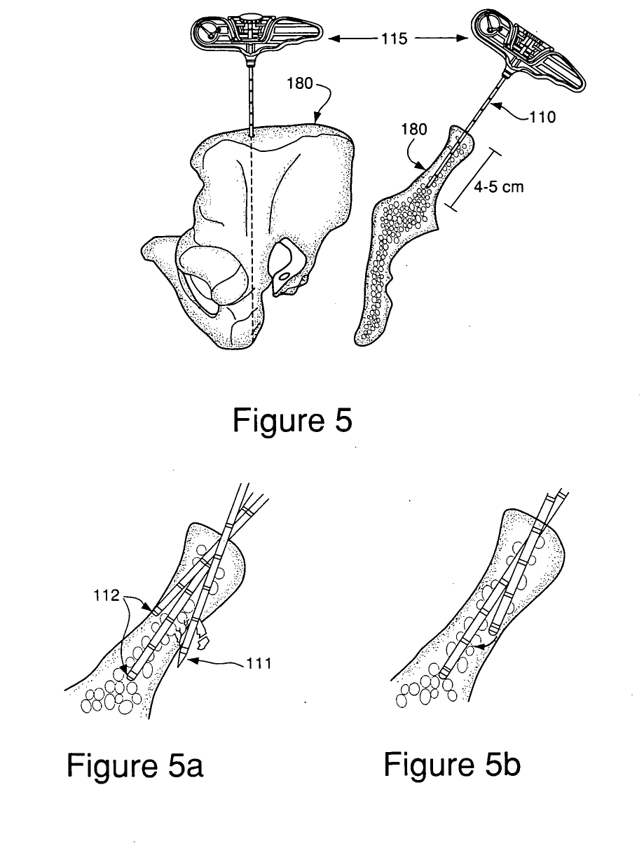

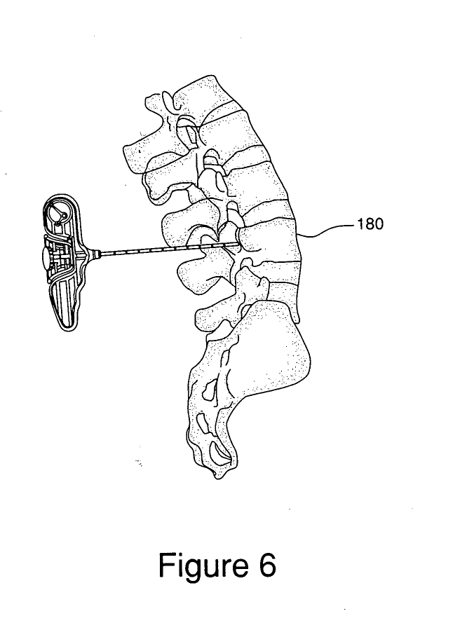

FIG. 5 depicts access to the iliac crest using an assembly in accordance with this invention. FIG. 5a and 5b show how a sharp stylet can puncture cortical bone remote from the site of entry; a very unfavorable occurrence. The navigation stylets as provided herein, having convex geometry at the tip, redirect from the interior surface of cortical bone back into the cancellous space. FIG. 6 illustrates how the present invention may also provide improved safety in accessing vertebral bodies. Thus, once the interior of a vertebra is accessed, a navigation stylet facilitates the avoidance of further perforation of the cortical bone with potentially crippling effect upon the spinal cord.

In addition, the cannula inlet port may be coupled to devices other than a syringe for use in a variety of procedures involving biopsy or aspiration, and the device assembly is not exclusive to bone marrow aspiration. The navigating stylet has been disclosed as a device that can be used to safely navigate within cortical bone boundaries and safely access cancellous bone marrow spaces. The bone boundaries can be the boundaries of the iliac crest, vertebral body, pedicles or other such bones as desired.

While the present invention has been disclosed for procedures involving the aspiration of bone marrow, the assembly could be used in other procedures in which it is desirable to safely gain access to the interior of bone. The present invention could be utilized in procedures involving the delivery of materials to bone (as opposed to procedures that involve the withdrawal of fluids such as bone marrow aspirate from the bone). For example, the present invention may find utility in procedures such as vertebroplasty or kyphoplasty in which it is necessary to gain access to the interior of vertebral bodies to inject hardenable materials such as bone cements, polymethylmethacrylate (PMMA), calcium phosphate injectables or other hardenable methacrylate based materials such as Cortoss® manufactured by Orthovita, Inc.®.

The sharp tip stylet is inserted into the cannula lumen and is mechanically interlocked by rotating the hub to engage the locking mechanism of the stylet into the notched void of the cannula handle. In FIG. 5, the trocar stylet 111 in the cannula 110 is used to perforate dense cortical bone here, in an iliac bone 180 in order to gain access to cancellous bone regions. Once the cortical bone has been perforated, the trocar stylet is removed by rotating the hub and withdrawing the stylet. The navigating stylet is then inserted into the lumen of the cannula and mechanically locked into place via the locking mechanism of the hub. This assembly allows the user to safely navigate within the cancellous bone, thus minimizing the risk of perforating the cortical bone boundaries as shown in FIG. 5a at 111. Rather, redirection within the cancellous space is had as seen in FIG. 5b. Once the cannula and bullet tip stylet are safely positioned within cancellous bone, the navigating stylet is unlocked and withdrawn from the cannula. A surgical syringe is then threaded onto the cannula Luer for bone marrow aspiration.

FIG. 6 depicts access of a vertebral body 180 by the assemblies of the present invention. This and other sites of the body may be so accessed with a reduced danger of undesired bone penetration.

What is claimed is:

1. A rigid, self re-directing stylet for navigating cancellous bone, the stylet having a proximal end for mating with a handle and a distal end having a convex geometry by reference to the plane normal to the axis of the stylet.

2. The stylet of claim 1 wherein the convex geometry is a portion of a sphere.

3. The stylet of claim 1 wherein the convex geometry is a portion of an oblate or an ovoid solid.

4. The stylet of claim 1 wherein the convex geometry comprises a plurality of bevels.

5. The stylet of claim 1 comprising stainless steel.

6. The stylet of claim 1 further comprising a hub comprising a locking tab on the proximal end.

7. The stylet of claim 3 wherein said hub comprises plastic.

8. A bone navigation system comprising:
a cannula having a proximal end and a distal end, said proximal end comprising a handle having finger recesses, Luer, and recess for receiving a locking tab, said distal end comprising fenestrations;
a trocar stylet having a hub comprising said locking tab and a hammering surface that mates with said handle; and
a rigid, self re-directing stylet for navigating cancellous bone after piercing cortical bone overlying the cancellous bone, the stylet having a proximal end for mating with a handle and a distal end having a convex geometry by reference to the plane normal to the axis of the stylet.

9. The system of claim 8 wherein the convex geometry is a portion of a sphere.

10. The system of claim 8 wherein the convex geometry is a portion of an oblate or an ovoid solid.

11. The system of claim 8 wherein the convex geometry comprises a plurality of bevels.

12. The system of claim 8 wherein said cannula and said stylets each comprise stainless steel.

13. The system of claim 8 wherein said cannula displays graduated markings for at least a portion of its length.

14. The system of claim 8 wherein said handle and the hubs comprise plastic.

15. The system of claim 8 wherein the redirecting stylet is solid.

16. The system of claim 8 comprising at least two sets of fenestrations, the first set including at least three fenestrations located about 20 mm from the distal end of the cannula and radially spaced about 120° apart on approximately the same cross-sectional plane; the second set of fenestrations including at least three fenestrations located about 10 mm from the distal end of the cannula on approximately the same cross-sectional plane; the first set of fenestrations being radially offset from the second set of fenestrations by about 90°.

17. A method for accessing cancellous bone within cortical bone comprising piercing the cortical bone with a first stylet, having a sharpened distal end, to create an access opening through the cortical bone and into the cancellous bone; and
inserting a second, generally rigid stylet into the access opening and into the space containing the cancellous bone, the second stylet having a distal end which is convex by reference to the plane normal to the axis of the stylet.
